(19) United States
(12) Patent Application Publication
(10) Pub. No.: US 2024/0366166 A1
(43) Pub. Date: Nov. 7, 2024

(54) TOMOGRAPHIC IMAGE CREATION DEVICE, TOMOGRAPHIC IMAGE CREATION METHOD, AND TOF-PET DEVICE

(71) Applicant: HAMAMATSU PHOTONICS K.K., Hamamatsu-shi, Shizuoka (JP)

(72) Inventors: Yuya ONISHI, Hamamatsu-shi, Shizuoka (JP); Ryosuke OTA, Hamamatsu-shi, Shizuoka (JP); Fumio HASHIMOTO, Hamamatsu-shi, Shizuoka (JP); Kibo OTE, Hamamatsu-shi, Shizuoka (JP)

(73) Assignee: HAMAMATSU PHOTONICS K.K., Hamamatsu-shi, Shizuoka (JP)

(21) Appl. No.: 18/683,279

(22) PCT Filed: Aug. 18, 2022
(86) PCT No.: PCT/JP2022/031259

§ 371 Date: Feb. 13, 2024

(30) Foreign Application Priority Data
Aug. 23, 2021 (JP) 2021-135572

(51) Int. Cl. A61B 6/00; A61B 6/03; A61B 6/42; G01T 1/20

(52) U.S. Cl. CPC A61B 6/5205; A61B 6/037; A61B 6/4241; G01T 1/20182

(57) ABSTRACT

A tomographic image creation apparatus includes a time difference calculation unit, a signal waveform processing unit, an error estimation unit, a γ-ray pair generation position calculation unit, and an image creation unit. The time difference calculation unit calculates, for each of γ-ray pair coincidence events, a time difference of a timing at which a value of each of a first signal and a second signal output from a signal waveform acquisition unit reaches a threshold value. The signal waveform processing unit relatively shifts a waveform of the first signal or a waveform of the second signal by the time difference in a direction approaching each other. The error estimation unit estimates an error of the time difference by a DNN based on the waveform of each of the first signal and the second signal after shifting.

### TECHNICAL FIELD

The present disclosure relates to a tomographic image creation apparatus, a tomographic image creation method, and a TOF-PET apparatus.

### BACKGROUND ART

A positron emission tomography (PET) apparatus includes a PET detection device including a large number of radiation detectors arranged around a measurement space, and a tomographic image creation apparatus for creating a tomographic image of a subject based on information of a large number of γ-ray pair coincidence events collected for the subject by the PET detection device. The subject into which a drug labeled with a positron emitting radionuclide is injected is placed in the measurement space of the PET detection device.

When a positron is emitted from the positron emitting radionuclide in a body of the subject, two γ-ray photons having energy of 511 keV are generated by pair annihilation of the positron and an electron. These two γ-ray photons (γ-ray pair) travel in opposite directions to each other, and are detected in coincidence by any two radiation detectors in the PET detection device. Further, the tomographic image creation apparatus performs required image reconstruction processing based on the collected information of the large number of γ-ray pair coincidence events, thereby creating an image representing a distribution of γ-ray pair generation positions (that is, the tomographic image of the subject).

In the PET apparatuses, a time-of-flight PET (TOF-PET) apparatus can detect the γ-ray pair generation position on a coincidence detection line connecting the two radiation detectors to each other based on a time difference between respective detection timings of the two radiation detectors which detect the pair of γ-rays in coincidence for each γ-ray pair coincidence event. Further, by detecting the γ-ray pair generation positions for the large number of γ-ray pair coincidence events, it is possible to create the image representing the distribution of the γ-ray pair generation positions (that is, the tomographic image of the subject).

Hereinafter, the above technique is referred to as a “comparative example 1”. In the TOF-PET apparatus, in order to create the tomographic image with high spatial resolution, it is desirable to obtain the time difference between the respective detection timings of the two radiation detectors, which detect the pair of γ-rays in coincidence, with high temporal resolution.

In a technique described in Non Patent Document 1 (hereinafter referred to as a “comparative example 2”), in the TOF-PET apparatus, respective waveforms of a first signal and a second signal output from the two radiation detectors which detect the pair of γ-rays in coincidence are input to a convolutional neural network (CNN), which is a type of a deep neural network (DNN). Further, the CNN estimates the time difference between the respective detection timings of the two radiation detectors which detect the pair of γ-rays in coincidence.

Compared to the comparative example 1, in the comparative example 2, the time difference between the respective detection timings of the two radiation detectors which detect the pair of γ-rays in coincidence can be obtained with high temporal resolution.

### CITATION LIST

### Non Patent Literature

Non Patent Document 1: E. Berg and S. Cherry, “Using convolutional neural networks to estimate time-of-flight from PET detector waveforms”, Phys. Med. Biol. 63 02LT01, 2018

### SUMMARY OF INVENTION

### Technical Problem

Compared to the comparative example 1, in the comparative example 2, the time difference between the respective detection timings of the two radiation detectors which detect the pair of γ-rays in coincidence can be obtained with high temporal resolution, and thus, it is expected that the tomographic image with high spatial resolution can be created. However, the present inventors have found that, in the comparative example 2, the amount of data required for training the CNN is enormous, and thus, there is a problem in that training of the CNN is not easy, and further, there is also a problem in that the created tomographic image is to be distorted.

An object of the present invention is to provide a tomographic image creation apparatus and a tomographic image creation method for creating a tomographic image of a subject using a DNN based on information of a plurality of γ-ray pair coincidence events collected by a PET detection device, capable of easily training the DNN, and capable of creating the tomographic image with small distortion. Further, an object of the present invention is to provide a TOF-PET apparatus including the above tomographic image creation apparatus and the PET detection device.

### Solution to Problem

An embodiment of the present invention is a tomographic image creation apparatus. The tomographic image creation apparatus is an apparatus for creating a tomographic image of a subject based on information of a plurality of γ-ray pair coincidence events collected for the subject placed in a measurement space of a PET detection device including a plurality of radiation detectors, and includes (1) a time difference calculation unit for calculating, for each of the plurality of γ-ray pair coincidence events, a time difference t_{led} of a timing at which a value of each of a first signal and a second signal output from two radiation detectors which detect a pair of γ-rays in coincidence out of the plurality of radiation detectors reaches a threshold value; (2) a signal waveform processing unit for relatively shifting a waveform of the first signal or a waveform of the second signal by the time difference t_{led} in a direction approaching each other in a time axis direction; (3) an error estimation unit for estimating an error tₑᵣᵣ of the time difference t_{led} by a deep neural network based on the waveform of each of the first signal and the second signal after shifting by the signal waveform processing unit; (4) a γ-ray pair generation position calculation unit for calculating a γ-ray pair generation position on a coincidence detection line connecting the two radiation detectors to each other based on the time difference t_{led} and the error tₑᵣᵣ; and (5) an image creation unit for creating the tomographic image of the subject based on the γ-ray pair generation position calculated by the γ-ray pair generation position calculation unit for each of the plurality of γ-ray pair coincidence events.

An embodiment of the present invention is a TOF-PET apparatus. The TOF-PET apparatus includes a PET detection device including a plurality of radiation detectors; and the tomographic image creation apparatus of the above configuration for creating the tomographic image of the subject based on the information of the plurality of γ-ray pair coincidence events collected for the subject placed in the measurement space of the PET detection device.

An embodiment of the present invention is a tomographic image creation method. The tomographic image creation method is a method for creating a tomographic image of a subject based on information of a plurality of γ-ray pair coincidence events collected for the subject placed in a measurement space of a PET detection device including a plurality of radiation detectors, and includes (1) a time difference calculation step of calculating, for each of the plurality of γ-ray pair coincidence events, a time difference t_{led} of a timing at which a value of each of a first signal and a second signal output from two radiation detectors which detect a pair of γ-rays in coincidence out of the plurality of radiation detectors reaches a threshold value; (2) a signal waveform processing step of relatively shifting a waveform of the first signal or a waveform of the second signal by the time difference t_{led} in a direction approaching each other in a time axis direction; (3) an error estimation step of estimating an error tₑᵣᵣ of the time difference t_{led} by a deep neural network based on the waveform of each of the first signal and the second signal after shifting by the signal waveform processing step; (4) a γ-ray pair generation position calculation step of calculating a γ-ray pair generation position on a coincidence detection line connecting the two radiation detectors to each other based on the time difference t_{led} and the error tₑᵣᵣ; and (5) an image creation step of creating the tomographic image of the subject based on the γ-ray pair generation position calculated by the γ-ray pair generation position calculation step for each of the plurality of γ-ray pair coincidence events.

### Advantageous Effects of Invention

According to the embodiments of the present invention, it is possible to create a tomographic image of a subject using a DNN based on information of a plurality of γ-ray pair coincidence events collected by a PET detection device, it is possible to easily train the DNN, and create the tomographic image with small distortion.

### BRIEF DESCRIPTION OF DRAWINGS

FIG. 1 is a diagram illustrating a configuration of a TOF-PET apparatus 1.

FIG. 2 is a diagram illustrating a processing content of a time difference calculation unit 12 of the TOF-PET apparatus 1.

FIG. 3 is a diagram illustrating a processing result of a signal waveform processing unit 13 of the TOF-PET apparatus 1.

FIG. 4 is a diagram illustrating a configuration of an experimental system used in an experiment performed for confirming an effect of an example in comparison with comparative examples.

FIG. 5 includes graphs each showing a distribution of a γ-ray pair generation position obtained in a comparative example 1.

FIG. 6 includes graphs each showing the distribution of the γ-ray pair generation position obtained in a comparative example 2A.

FIG. 7 includes graphs each showing the distribution of the γ-ray pair generation position obtained in a comparative example 2B.

FIG. 8 includes graphs each showing the distribution of the γ-ray pair generation position obtained in the example.

FIG. 9 is a table showing peak positions of the distributions of the γ-ray pair generation positions obtained respectively in the comparative example 1, the comparative example 2A, the comparative example 2B, and the example.

FIG. 10 is a table showing full widths at half maximum of the distributions of the γ-ray pair generation positions obtained respectively in the comparative example 1, the comparative example 2A, the comparative example 2B, and the example.

FIG. 11 is a diagram illustrating a position where a positron emitting radionuclide 3 is to be disposed in a measurement space of a PET detection device 20 for collecting training data in the comparative example 2 when there is no performance variation in a plurality of radiation detectors of the PET detection device 20.

FIG. 12 is a diagram illustrating the position where the positron emitting radionuclide 3 is to be disposed in the measurement space of the PET detection device 20 for collecting the training data in the comparative example 2 when there is performance variation in the plurality of radiation detectors of the PET detection device 20.

FIG. 13 is a diagram illustrating the position where the positron emitting radionuclide 3 is to be disposed in the measurement space of the PET detection device 20 for collecting the training data in the present embodiment when there is no performance variation in the plurality of radiation detectors of the PET detection device 20.

FIG. 14 is a diagram illustrating the position where the positron emitting radionuclide 3 is to be disposed in the measurement space of the PET detection device 20 for collecting the training data in the present embodiment when there is performance variation in the plurality of radiation detectors of the PET detection device 20.

### DESCRIPTION OF EMBODIMENTS

Hereinafter, embodiments of a tomographic image creation apparatus, a tomographic image creation method, and a TOF-PET apparatus will be described in detail with reference to the accompanying drawings. In the description of the drawings, the same elements will be denoted by the same reference signs, and redundant description will be omitted. The present invention is not limited to these examples.

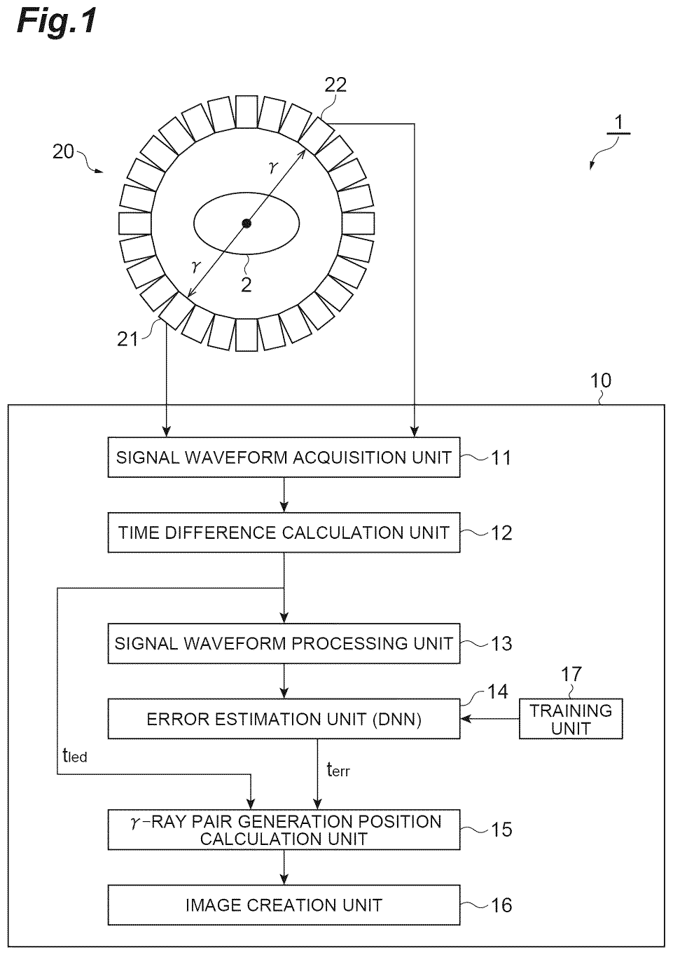

FIG. 1 is a diagram illustrating a configuration of a TOF-PET apparatus 1. The TOF-PET apparatus 1 includes a tomographic image creation apparatus 10, and a PET detection device 20.

The PET detection device 20 includes a large number of radiation detectors arranged in a ring shape around a measurement space in which a subject 2 is to be placed. The subject 2 into which a drug labeled with a positron emitting radionuclide is injected is placed in the measurement space of the PET detection device 20. When a positron is emitted from the positron emitting radionuclide in a body of the subject 2, two γ-ray photons having energy of 511 keV are generated by pair annihilation of the positron and an electron.

These two γ-ray photons (γ-ray pair) travel in opposite directions to each other, and are detected in coincidence by any two radiation detectors 21 and 22 out of the plurality of radiation detectors included in the PET detection device 20. Each of the plurality of radiation detectors of the PET detection device 20 outputs a pulse signal in response to a γ-ray detection event. In addition, in FIG. 1, flight paths of a certain pair of γ-rays generated at a certain position (γ-ray pair generation position) in the body of the subject 2 are illustrated by arrows, and two radiation detectors which detect the pair of γ-rays out of the plurality of radiation detectors are illustrated as the radiation detectors 21 and 22.

The tomographic image creation apparatus 10 creates a tomographic image of the subject 2 based on information of a large number of γ-ray pair coincidence events collected for the subject 2 placed in the measurement space of the PET detection device 20. The tomographic image creation apparatus 10 includes a signal waveform acquisition unit 11, a time difference calculation unit 12, a signal waveform processing unit 13, an error estimation unit 14, a γ-ray pair generation position calculation unit 15, an image creation unit 16, and a training unit 17.

The signal waveform acquisition unit 11 is connected to each of the plurality of radiation detectors of the PET detection device 20 via a signal line, and inputs the pulse signal output from each of the plurality of radiation detectors in response to the γ-ray detection event. In addition, in FIG. 1, the signal lines between the signal waveform acquisition unit 11 and the two radiation detectors 21 and 22 which detect the certain pair of γ-rays out of the plurality of radiation detectors in the PET detection device 20 are illustrated, and the signal lines between the signal waveform acquisition unit 11 and the other radiation detectors are not illustrated for simplification of illustration.

The signal waveform acquisition unit 11 detects the γ-ray pair coincidence event caused by any two radiation detectors out of the plurality of radiation detectors based on the pulse signal output from each of the plurality of radiation detectors of the PET detection device 20 in response to the γ-ray detection event, and identifies the two radiation detectors. Further, the signal waveform acquisition unit 11 outputs the waveform of each of the pulse signals (first signal, second signal) output from the two radiation detectors which detect the pair of γ-rays in coincidence to the time difference calculation unit 12, for each of the large number of γ-ray pair coincidence events.

The time difference calculation unit 12 inputs the waveform of each of the first signal and the second signal output from the signal waveform acquisition unit 11, for each of the large number of γ-ray pair coincidence events. Further, the time difference calculation unit 12 calculates a time difference t_{led} of a timing at which a value of each of the first signal and the second signal, being the pulse signals, reaches a threshold value.

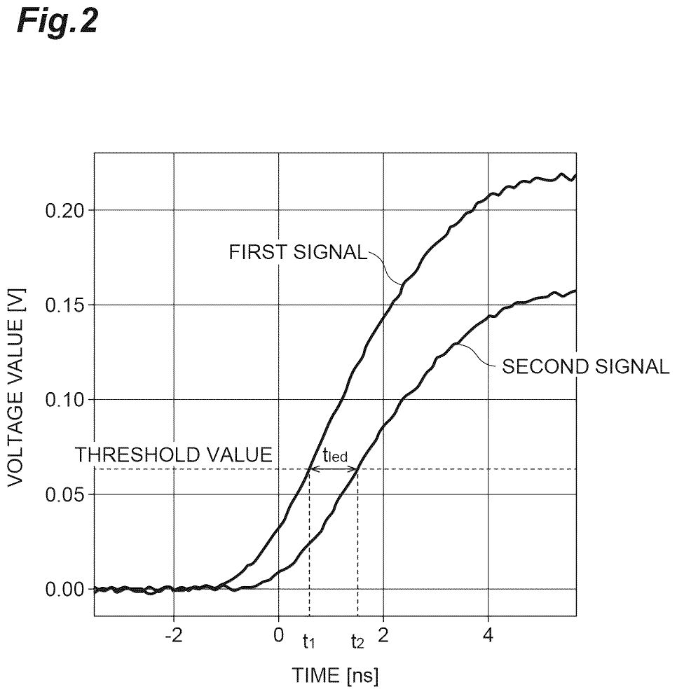

FIG. 2 is a diagram illustrating a processing content of the time difference calculation unit 12. The time difference calculation unit 12 calculates a timing t₁ at which the value of the first signal reaches the threshold value, and calculates a timing t₂ at which the value of the second signal reaches the threshold value, and further, calculates the time difference t_{led} between these timings t₁ and t₂. In addition, the above processing is called lead edge discriminator (LED), and is performed also in the comparative example 1.

The signal waveform processing unit 13 relatively shifts the waveform of the first signal or the waveform of the second signal by the time difference t_{led} in a direction approaching each other in a time axis direction. The signal waveform processing unit 13 may shift any one of the waveform of the first signal and the waveform of the second signal toward the other in the time axis direction, or may shift both the waveform of the first signal and the waveform of the second signal toward each other in the time axis direction. In addition, when the time difference t_{led} is 0, it is not necessary to shift both the waveform of the first signal and the waveform of the second signal in the time axis direction.

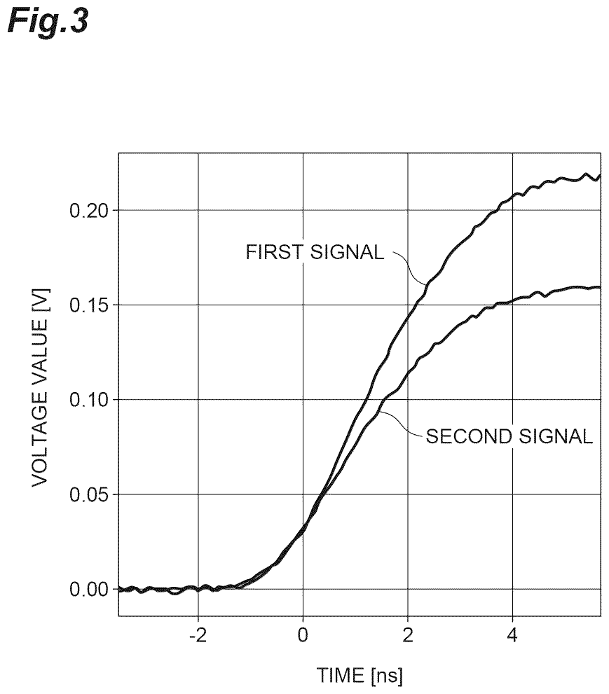

FIG. 3 is a diagram illustrating a processing result of the signal waveform processing unit 13. The timings at which the respective values of the first signal and the second signal after shifting by the signal waveform processing unit 13 reach the threshold value are expected to be equal to each other. However, in practice, the time difference t_{led} obtained by the time difference calculation unit 12 may include an error, and thus, these timings may not become equal to each other. In addition, it is considered that the error included in the time difference t_{led} obtained by the time difference calculation unit 12 does not depend on the γ-ray pair generation position.

The error estimation unit 14 estimates an error tₑᵣᵣ included in the time difference t_{led} calculated by the time difference calculation unit 12 based on the respective waveforms (FIG. 3) of the first signal and the second signal after the shifting by the signal waveform processing unit 13. In the above estimation of the error tₑᵣᵣ, a DNN is used, and preferably, a CNN, which is a type of the DNN, is used.

The γ-ray pair generation position calculation unit 15 calculates a more accurate time difference tₑₛₜ (=t_{led}-tₑᵣᵣ) based on the time difference t_{led} calculated by the time difference calculation unit 12 and the error tₑᵣᵣ estimated by the error estimation unit 14. Further, based on the above time difference tₑₛₜ, the γ-ray pair generation position calculation unit 15 calculates a γ-ray pair generation position on a coincidence detection line connecting the two radiation detectors which detect the pair of γ-rays in coincidence.

The image creation unit 16 creates the tomographic image of the subject 2 based on the γ-ray pair generation position calculated by the γ-ray pair generation position calculation unit 15 for each of the large number of γ-ray pair coincidence events.

The training unit 17 trains the DNN in the error estimation unit 14 based on the information of the large number of γ-ray pair coincidence events collected for the positron emitting radionuclide placed in the measurement space of the PET detection device 20 instead of the subject 2. The training unit 17 trains the DNN, for each of the plurality of γ-ray pair coincidence events, using the respective waveforms (FIG. 3) of the first signal and the second signal after the shifting by the signal waveform processing unit 13 as input data to the DNN, and further, using a difference between the time difference t_{led} calculated by the time difference calculation unit 12 and a true time difference based on a position of the positron emitting radionuclide as teaching data.

The tomographic image creation method using the tomographic image creation apparatus 10 described above includes a signal waveform acquisition step performed by the signal waveform acquisition unit 11, a time difference calculation step performed by the time difference calculation unit 12, a signal waveform processing step performed by the signal waveform processing unit 13, an error estimation step performed by the error estimation unit 14, a γ-ray pair generation position calculation step performed by the γ-ray pair generation position calculation unit 15, an image creation step performed by the image creation unit 16, and a training step performed by the training unit 17.

That is, in the signal waveform acquisition step, the pulse signal output from each of the plurality of radiation detectors of the PET detection device 20 in response to the γ-ray detection event is input. In the time difference calculation step, for each of the plurality of γ-ray pair coincidence events, the time difference t_{led} of the timing at which the value of each of the first signal and the second signal output from the two radiation detectors which detect the pair of γ-rays in coincidence out of the plurality of radiation detectors reaches the threshold value is calculated. In the signal waveform processing step, the waveform of the first signal or the waveform of the second signal is relatively shifted by the time difference t_{led} in the direction approaching each other in the time axis direction.

In the error estimation step, the error tₑᵣᵣ of the time difference t_{led} is estimated by the DNN based on the waveform of each of the first signal and the second signal after the shifting by the signal waveform processing step. In the γ-ray pair generation position calculation step, the γ-ray pair generation position on the coincidence detection line connecting the two radiation detectors to each other is calculated based on the time difference t_{led} and the error tₑᵣᵣ. In the image creation step, the tomographic image of the subject 2 is created based on the γ-ray pair generation position calculated by the γ-ray pair generation position calculation step for each of the plurality of γ-ray pair coincidence events in the PET detection device 20.

In the training step, the DNN is trained based on the information of the plurality of γ-ray pair coincidence events collected for the positron emitting radionuclide placed in the measurement space of the PET detection device 20. In addition, when the DNN has already been trained, the training step and the training unit 17 are not necessary. However, even when the DNN has already been trained, the training step and the training unit 17 may be provided when training is further performed in order to enable estimation with higher accuracy.

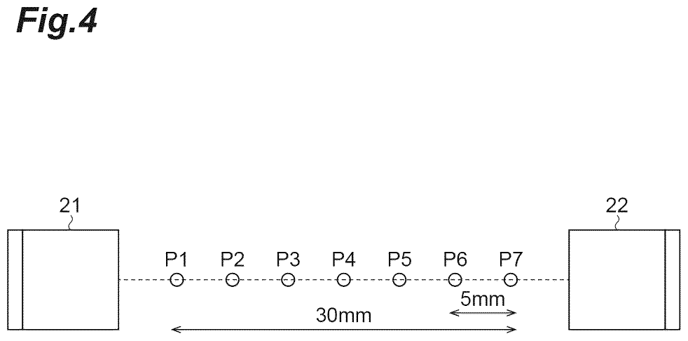

Next, results of an experiment performed for confirming effects of the present embodiment in comparison with the comparative examples will be described. FIG. 4 is a diagram illustrating a configuration of an experimental system. In this experiment, the positron emitting radionuclide (²²Na) is sequentially placed at each of seven positions P1 to P7 spaced at a 5 mm pitch on a line (corresponding to the coincidence detection line) connecting the two radiation detectors 21 and 22 to each other.

Each of the radiation detectors 21 and 22 includes an LYSO (Cerium Doped Lutetium Yttrium Orthosilicate) scintillator provided on a light receiving surface of an MPPC (Multi-Pixel Photon Counter). The MPPC (registered trademark) includes a two-dimensional array of a plurality of pixels, in which each pixel is formed by connecting a quenching resistor to an avalanche photodiode operating in Geiger mode, and can perform high speed and high sensitivity photodetection. A size of the light receiving surface of the MPPC is 3 mm×3 mm. A size of the LYSO scintillator is 3 mm×3 mm×10 mm thickness.

In the comparative example 1, for each of the γ-ray pair coincidence events, the γ-ray pair generation position is calculated based on the time difference t_{led} calculated by the time difference calculation unit 12.

Both the comparative example 2A and the comparative example 2B correspond to the technique (the comparative example 2) described in Non Patent Document 1 described above, but are different in a database used for training the CNN. In the comparative examples 2A and 2B, for each of the γ-ray pair coincidence events, the time difference between the signals is estimated by the CNN based on the respective waveforms (FIG. 2) of the first signal and the second signal acquired by the signal waveform acquisition unit 11, and the γ-ray pair generation position is obtained based on the estimated time difference.

In the comparative example 2A, in training the CNN, the respective waveforms (FIG. 2) of the first signal and the second signal acquired by the signal waveform acquisition unit 11 when the positron emitting radionuclide is placed at each of the seven positions P1 to P7 are used as the input data to the CNN, and the true time difference based on the position at which the positron emitting radionuclide is placed is used as the teaching data.

In the comparative example 2B, in training the CNN, the respective waveforms (FIG. 2) of the first signal and the second signal acquired by the signal waveform acquisition unit 11 when the positron emitting radionuclide is placed at each of the six positions P1 to P4, P6, and P7 excluding the position P5 are used as the input data to the CNN, and the true time difference based on the position at which the positron emitting radionuclide is placed is used as the teaching data.

In the example, the γ-ray pair generation position is calculated by the tomographic image creation apparatus 10 or the tomographic image creation method of the present embodiment described above. In the example, in training the CNN, the respective waveforms (FIG. 3) of the first signal and the second signal after the shifting by the signal waveform processing unit 13 when the positron emitting radionuclide is placed only at the position P4 out of the seven positions P1 to P7 are used as the input data to the CNN, and the difference between the time difference t_{led} calculated by the time difference calculation unit 12 and the true time difference based on the position P4 of the positron emitting radionuclide is used as the teaching data.

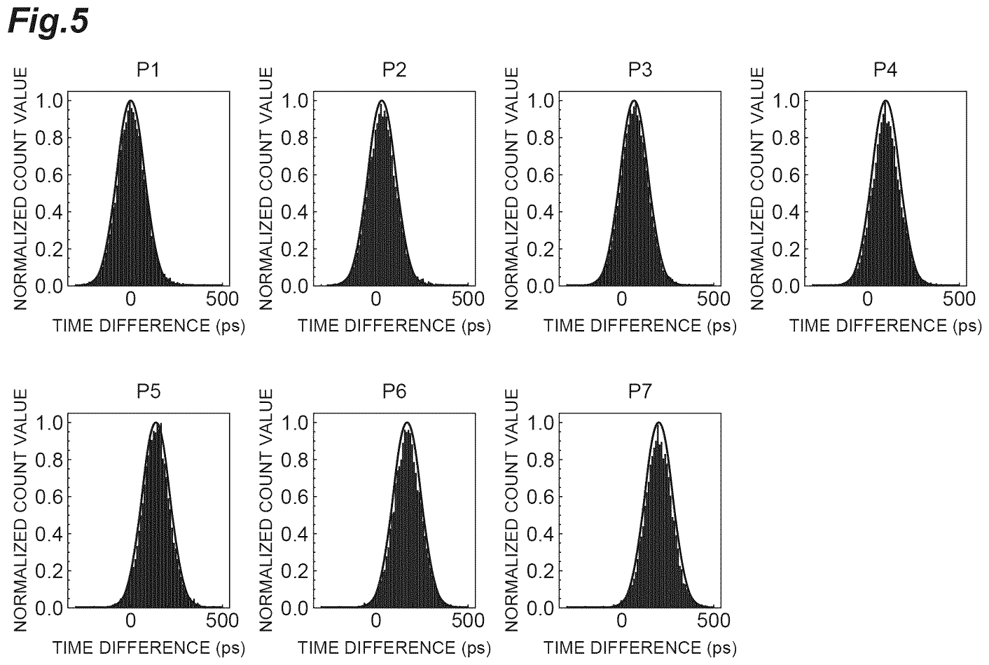

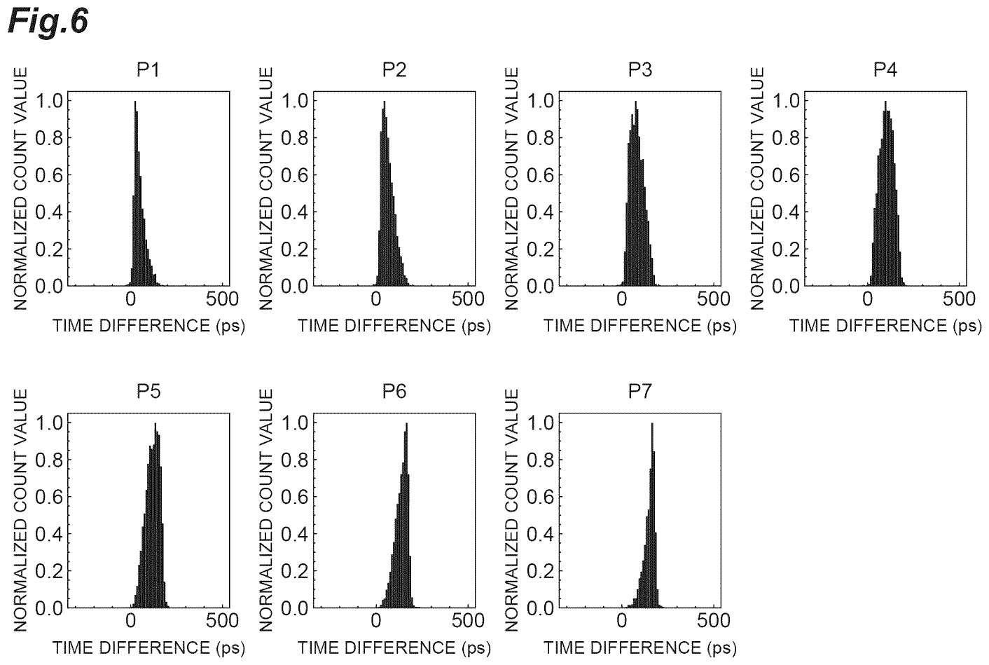

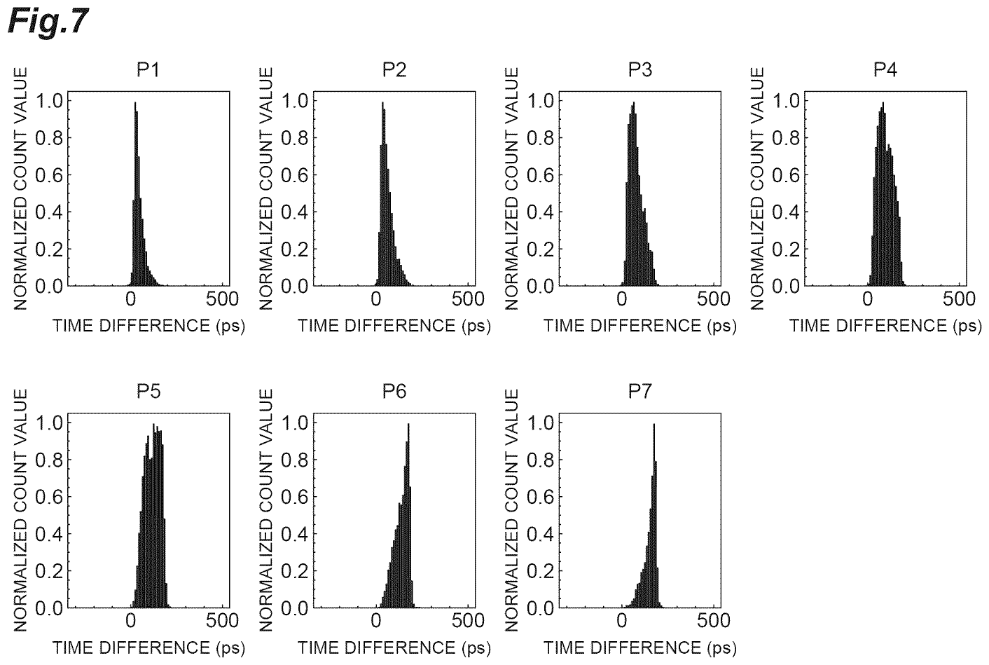

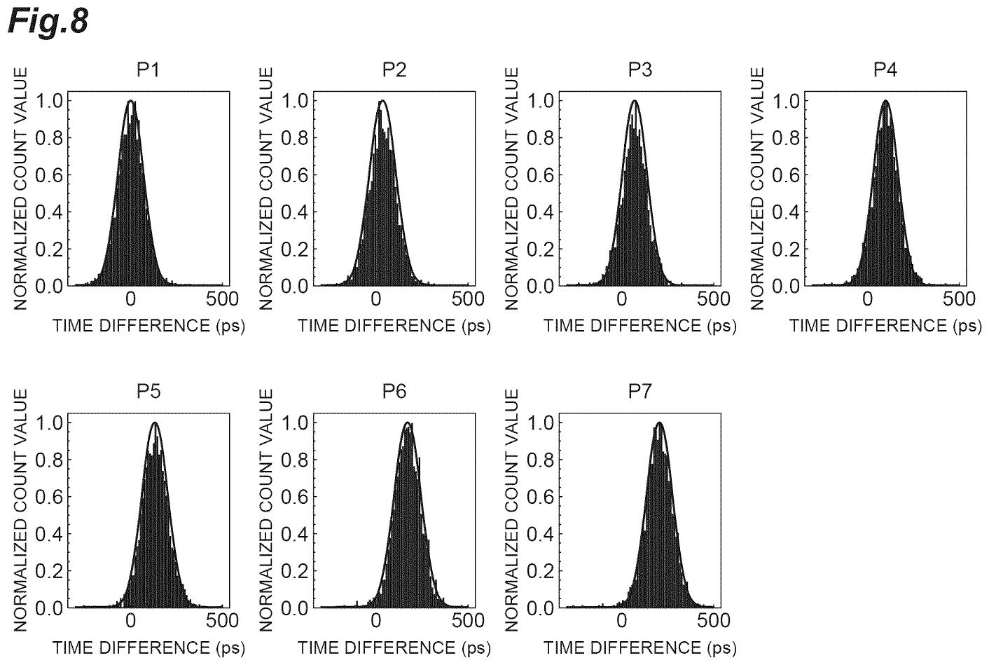

FIG. 5 includes graphs each showing the distribution of the γ-ray pair generation position obtained in the comparative example 1. FIG. 6 includes graphs each showing the distribution of the γ-ray pair generation position obtained in the comparative example 2A. FIG. 7 includes graphs each showing the distribution of the γ-ray pair generation position obtained in the comparative example 2B. FIG. 8 includes graphs each showing the distribution of the γ-ray pair generation position obtained in the example. FIG. 5 to FIG. 8 show the shapes of the distributions of the γ-ray pair generation positions obtained when the positron emitting radionuclide is placed at each of the seven positions P1 to P7.

From these diagrams, the following can be said for the shape of the distribution of the obtained γ-ray pair generation position. In the comparative example 1 (FIG. 5) and the example (FIG. 8), the distribution of the obtained γ-ray pair generation position is substantially bilaterally symmetrical with a peak position as a center. On the other hand, in the comparative example 2A (FIG. 6), the distribution of the γ-ray pair generation position obtained when the positron emitting radionuclide is placed at the central position P4 is substantially bilaterally symmetrical with the peak position as the center, but the distribution of the γ-ray pair generation position obtained when the positron emitting radionuclide is placed at the position other than the central position P4 is not bilaterally symmetrical with the peak position as the center, and the peak position is shifted to the side far from the central position P4.

In the comparative example 2B (FIG. 7), the following can be said, in addition to the above-described tendency of the comparative example 2A. In the comparative example 2B in which the data obtained when the positron emitting radionuclide is placed at the position P5 is not used for training the CNN, two peaks appear in the distribution of the γ-ray pair generation position obtained when the positron emitting radionuclide is placed at the position P5. Further, in the comparative example 2B, the distribution of the γ-ray pair generation position obtained when the positron emitting radionuclide is placed at the central position P4 is not bilaterally symmetrical with the peak position as the center, and the peak position is shifted to the side of the position P3.

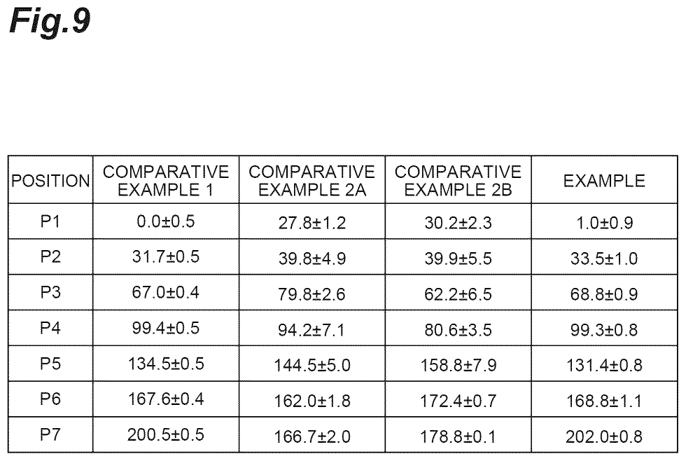

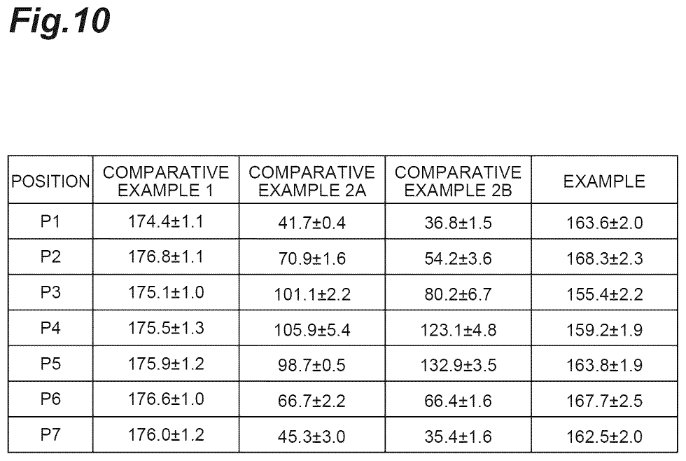

FIG. 9 is a table showing peak positions of the distributions of the γ-ray pair generation positions obtained respectively in the comparative example 1, the comparative example 2A, the comparative example 2B, and the example. FIG. 10 is a table showing full widths at half maximum of the distributions of the γ-ray pair generation positions obtained respectively in the comparative example 1, the comparative example 2A, the comparative example 2B, and the example. FIG. 9 and FIG. 10 are obtained from the shapes of the distributions of the γ-ray pair generation positions shown in FIG. 5 to FIG. 8, and show the peak positions or the full widths at half maximum of the distributions of the γ-ray pair generation positions obtained when the positron emitting radionuclide is placed at each of the seven positions P1 to P7 in time (unit: ps).

The seven positions P1 to P7 are separated with a 5 mm pitch, and thus, ideally, the peak positions of the distributions of the obtained γ-ray pair generation positions are to be separated with a 33 ps pitch. As shown in FIG. 9, in the example and the comparative example 1, the peak positions of the distributions of the obtained γ-ray pair generation positions are substantially ideally separated with the 33 ps pitch. On the other hand, in the comparative example 2A and the comparative example 2B, the pitch of the peak positions of the distributions of the obtained γ-ray pair generation positions is different from the ideal case, and in particular, the farther from the central position P4, the narrower the pitch of the peak positions of the distributions of the obtained γ-ray pair generation positions is.

As shown in FIG. 10, the full widths at half maximum of the distributions of the obtained γ-ray pair generation positions are the narrowest in the comparative example 2A and the comparative example 2B, and the next narrowest in the example. That is, temporal resolution of the detection of the γ-ray pair generation position is the highest in the comparative example 2A and the comparative example 2B, and the next highest in the example. The full width at half maximum of the distribution of the γ-ray pair generation position obtained when the positron emitting radionuclide is placed at the central position P4 is 175.5 ps in the comparative example 1, and is 159.2 ps in the example, and thus, temporal resolution is higher in the example than in the comparative example 1.

The following can be said from the experimental results shown in FIG. 5 to FIG. 10. In the present embodiment, similarly to the comparative example 1, the pitch of the peak positions of the distributions of the γ-ray pair generation positions obtained for the positron emitting radionuclide placed at each position with the constant pitch is also substantially constant, and thus, it is possible to create the tomographic image with small distortion. In the present embodiment, temporal resolution of the detection of the γ-ray pair generation position is higher than that in the comparative example 1, and the tomographic image having high spatial resolution can be acquired.

In the comparative example 2 (2A, 2B), although the γ-ray pair generation position can be obtained with high temporal resolution, the pitch of the peak positions of the distributions of the γ-ray pair generation positions obtained for the positron emitting radionuclide placed at each position with the constant pitch is not constant, and thus, the created tomographic image is distorted.

In the comparative example 2, it is considered that the tomographic image with small distortion can be created by training the CNN using the training data acquired by densely placing the positron emitting radionuclide at a large number of positions over a range wider than a space occupied by the subject (in some cases, a range wider than the measurement space surrounded by the large number of radiation detectors), however, it is difficult to prepare such a large amount of training data, and it is also difficult to train the CNN.

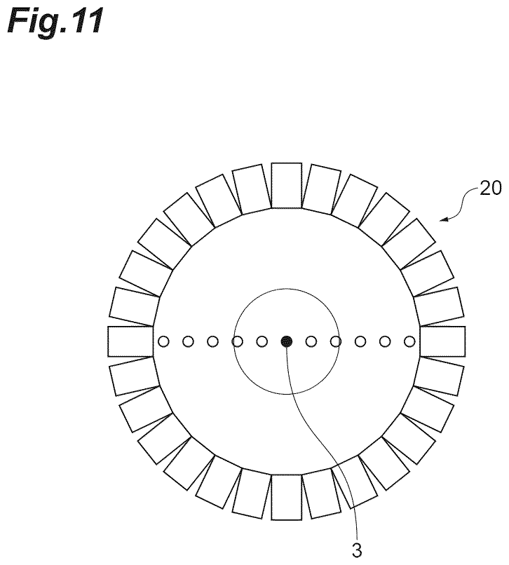

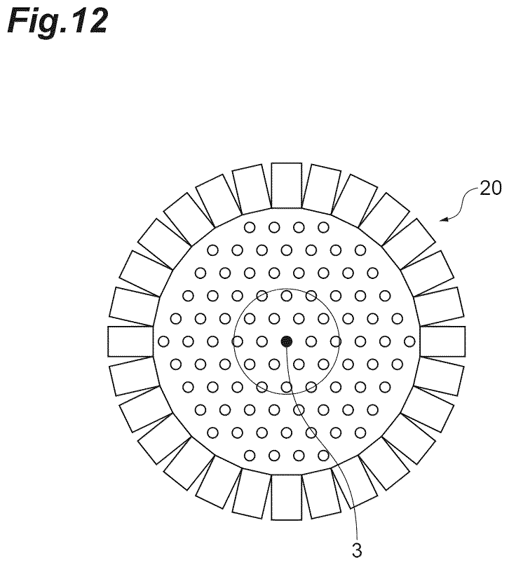

FIG. 11 and FIG. 12 are diagrams each illustrating the position at which the positron emitting radionuclide 3 is to be disposed in the measurement space of the PET detection device 20 for collecting the training data in the comparative example 2. FIG. 11 illustrates a case in which there is no performance variation between the plurality of radiation detectors of the PET detection device 20. In this case, it is necessary to collect the training data by densely placing the positron emitting radionuclide 3 at a large number of positions on a straight line extending in a radial direction.

FIG. 12 illustrates a case in which there is performance variation between the plurality of radiation detectors of the PET detection device 20. In this case, it is necessary to collect the training data by densely placing the positron emitting radionuclide 3 at a large number of grid-shaped positions. In the comparative example 2, a field of view of the apparatus is to be limited in any case.

On the other hand, in the present embodiment, it is only necessary to train the DNN by using the training data acquired by placing the positron emitting radionuclide at a smaller number of positions than in the comparative example 2 (2A, 2B), and thus, the DNN can be easily trained.

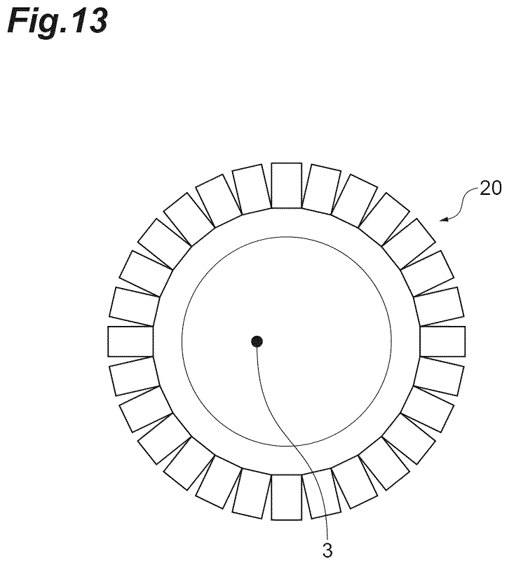

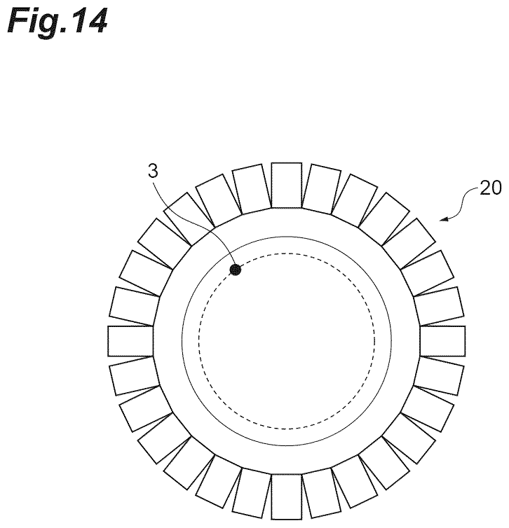

FIG. 13 and FIG. 14 are diagrams each illustrating the position at which the positron emitting radionuclide 3 is to be disposed in the measurement space of the PET detection device 20 for collecting the training data in the present embodiment. FIG. 13 illustrates a case in which there is no performance variation between the plurality of radiation detectors of the PET detection device 20. In this case, the training data may be collected by placing the positron emitting radionuclide 3 at one arbitrary position in the measurement space.

FIG. 14 illustrates a case in which there is performance variation between the plurality of radiation detectors of the PET detection device 20. In this case, for example, the training data may be collected for all radiation detector pairs while rotating the positron emitting radionuclide 3 around the center axis in the measurement space. In the present embodiment, a field of view of the apparatus is not limited as in the comparative example 2.

The tomographic image creation apparatus, the tomographic image creation method, and the TOF-PET apparatus according to the present invention are not limited to the embodiments and configuration examples described above, and various modifications are possible.

The tomographic image creation apparatus of the above embodiment is an apparatus for creating a tomographic image of a subject based on information of a plurality of γ-ray pair coincidence events collected for the subject placed in a measurement space of a PET detection device including a plurality of radiation detectors, and includes (1) a time difference calculation unit for calculating, for each of the plurality of γ-ray pair coincidence events, a time difference t_{led} between timings at which respective values of a first signal and a second signal output from two radiation detectors which detect a pair of γ-rays in coincidence out of the plurality of radiation detectors reach a threshold value; (2) a signal waveform processing unit for relatively shifting a waveform of the first signal or a waveform of the second signal by the time difference t_{led} in a direction approaching each other in a time axis direction; (3) an error estimation unit for estimating an error tₑᵣᵣ of the time difference t_{led} by a deep neural network based on the waveform of each of the first signal and the second signal after shifting by the signal waveform processing unit; (4) a γ-ray pair generation position calculation unit for calculating a γ-ray pair generation position on a coincidence detection line connecting the two radiation detectors to each other based on the time difference t_{led} and the error tₑᵣᵣ; and (5) an image creation unit for creating the tomographic image of the subject based on the γ-ray pair generation position calculated by the γ-ray pair generation position calculation unit for each of the plurality of γ-ray pair coincidence events.

The above tomographic image creation apparatus may further include a training unit for training the deep neural network, based on the information of the plurality of γ-ray pair coincident events collected for a positron emitting radionuclide placed in the measurement space, for each of the plurality of γ-ray pair coincident events, using the waveform of each of the first signal and the second signal after shifting by the signal waveform processing unit as input data to the deep neural network, and using a difference between the time difference t_{led} calculated by the time difference calculation unit and a true time difference based on a position of the positron emitting radionuclide as teaching data.

The TOF-PET apparatus of the above embodiment includes a PET detection device including a plurality of radiation detectors; and the tomographic image creation apparatus of the above configuration for creating the tomographic image of the subject based on the information of the plurality of γ-ray pair coincidence events collected for the subject placed in the measurement space of the PET detection device.

The tomographic image creation method of the above embodiment is a method for creating a tomographic image of a subject based on information of a plurality of γ-ray pair coincidence events collected for the subject placed in a measurement space of a PET detection device including a plurality of radiation detectors, and includes (1) a time difference calculation step of calculating, for each of the plurality of γ-ray pair coincidence events, a time difference t_{led} between timings at which respective values of a first signal and a second signal output from two radiation detectors which detect a pair of γ-rays in coincidence out of the plurality of radiation detectors reach a threshold value; (2) a signal waveform processing step of relatively shifting a waveform of the first signal or a waveform of the second signal by the time difference t_{led} in a direction approaching each other in a time axis direction; (3) an error estimation step of estimating an error tₑᵣᵣ of the time difference t_{led} by a deep neural network based on the waveform of each of the first signal and the second signal after shifting by the signal waveform processing step; (4) a γ-ray pair generation position calculation step of calculating a γ-ray pair generation position on a coincidence detection line connecting the two radiation detectors to each other based on the time difference t_{led} and the error tₑᵣᵣ; and (5) an image creation step of creating the tomographic image of the subject based on the γ-ray pair generation position calculated by the γ-ray pair generation position calculation step for each of the plurality of γ-ray pair coincidence events.

The above tomographic image creation method may further include a training step of training the deep neural network, based on the information of the plurality of γ-ray pair coincident events collected for a positron emitting radionuclide placed in the measurement space, for each of the plurality of γ-ray pair coincident events, using the waveform of each of the first signal and the second signal after shifting by the signal waveform processing step as input data to the deep neural network, and using a difference between the time difference t_{led} calculated by the time difference calculation step and a true time difference based on a position of the positron emitting radionuclide as teaching data.

### INDUSTRIAL APPLICABILITY

The present invention can be used as a tomographic image creation apparatus, a tomographic image creation method, and a TOF-PET apparatus for creating a tomographic image of a subject using a DNN based on information of a plurality of γ-ray pair coincidence events collected by a PET detection device, capable of easily training the DNN, and capable of creating the tomographic image with small distortion.

### REFERENCE SIGNS LIST

TOF-PET apparatus, 2—subject, 3—positron emitting radionuclide, 10—tomographic image creation apparatus, 11—signal waveform acquisition unit, 12—time difference calculation unit, 13—signal waveform processing unit, 14—error estimation unit, 15—γ-ray pair generation position calculation unit, 16—image creation unit, 17—training unit, 20—PET detection device, 21, 22—radiation detector.

1. A tomographic image creation apparatus for creating a tomographic image of a subject based on information of a plurality of γ-ray pair coincidence events collected for the subject placed in a measurement space of a PET detection device including a plurality of radiation detectors, the apparatus comprising:
a time difference calculation unit configured to calculate, for each of the plurality of γ-ray pair coincidence events, a time difference t_{led} of a timing at which a value of each of a first signal and a second signal output from two radiation detectors which detect a pair of γ-rays in coincidence out of the plurality of radiation detectors reaches a threshold value;
a signal waveform processing unit configured to relatively shift a waveform of the first signal or a waveform of the second signal by the time difference t_{led} in a direction approaching each other in a time axis direction;
an error estimation unit configured to estimate an error tₑᵣᵣ of the time difference t_{led} by a deep neural network based on the waveform of each of the first signal and the second signal after shifting by the signal waveform processing unit;
a γ-ray pair generation position calculation unit configured to calculate a γ-ray pair generation position on a coincidence detection line connecting the two radiation detectors to each other based on the time difference t_{led} and the error tₑᵣᵣ; and
an image creation unit configured to create the tomographic image of the subject based on the γ-ray pair generation position calculated by the γ-ray pair generation position calculation unit for each of the plurality of γ-ray pair coincidence events.

2. The tomographic image creation apparatus according to claim 1, further comprising a training unit configured to train the deep neural network, based on the information of the plurality of γ-ray pair coincident events collected for a positron emitting radionuclide placed in the measurement space, for each of the plurality of γ-ray pair coincident events, using the waveform of each of the first signal and the second signal after shifting by the signal waveform processing unit as input data to the deep neural network, and using a difference between the time difference t_{led} calculated by the time difference calculation unit and a true time difference based on a position of the positron emitting radionuclide as teaching data.

3. A TOF-PET apparatus comprising:
a PET detection device including a plurality of radiation detectors; and
the tomographic image creation apparatus according to claim 1 configured to create the tomographic image of the subject based on the information of the plurality of γ-ray pair coincidence events collected for the subject placed in the measurement space of the PET detection device.

4. A tomographic image creation method for creating a tomographic image of a subject based on information of a plurality of γ-ray pair coincidence events collected for the subject placed in a measurement space of a PET detection device including a plurality of radiation detectors, the method comprising:
performing a time difference calculation of calculating, for each of the plurality of γ-ray pair coincidence events, a time difference t_{led} of a timing at which a value of each of a first signal and a second signal output from two radiation detectors which detect a pair of γ-rays in coincidence out of the plurality of radiation detectors reaches a threshold value;
performing a signal waveform processing of relatively shifting a waveform of the first signal or a waveform of the second signal by the time difference t_{led} in a direction approaching each other in a time axis direction;
performing an error estimation of estimating an error tₑᵣᵣ of the time difference t_{led} by a deep neural network based on the waveform of each of the first signal and the second signal after shifting by the signal waveform processing;
performing a γ-ray pair generation position calculation of calculating a γ-ray pair generation position on a coincidence detection line connecting the two radiation detectors to each other based on the time difference t_{led} and the error tₑᵣᵣ; and
performing an image creation of creating the tomographic image of the subject based on the γ-ray pair generation position calculated by the γ-ray pair generation position calculation for each of the plurality of γ-ray pair coincidence events.

5. The tomographic image creation method according to claim 4, further comprising performing a training of training the deep neural network, based on the information of the plurality of γ-ray pair coincident events collected for a positron emitting radionuclide placed in the measurement space, for each of the plurality of γ-ray pair coincident events, using the waveform of each of the first signal and the second signal after shifting by the signal waveform processing as input data to the deep neural network, and using a difference between the time difference t_{led} calculated by the time difference calculation and a true time difference based on a position of the positron emitting radionuclide as teaching data.
